(19) United States
(12) Patent Application Publication
(10) Pub. No.: US 2024/0066305 A1
(43) Pub. Date: Feb. 29, 2024

(54) Methods and Electrical Stimulators for Interferential Stimulation using Axial Bias Stimulation Fields

(71) Applicant: Neuromodulation Specialists, LTD, London (GB)

(72) Inventors: Thomas L. Yearwood, London (GB); William J. Carroll, LaCenter, WA (US)

(21) Appl. No.: 17/900,559

(22) Filed: Aug. 31, 2022

(51) Int. Cl. A61N 1/36; A61N 1/05

(52) U.S. Cl. CPC A61N 1/36185; A61N 1/0551; A61N 1/36062

(57) ABSTRACT

An example method for electrical stimulation of a subject includes creating multiple circuits using implantable electrodes positioned in the subject, transmitting a signal of a first frequency through a first circuit of the multiple circuits and the first circuit generates a first electrical field, and transmitting a signal of a second frequency through a second circuit of the multiple circuits and the second circuit generates a second electrical field. The implantable electrodes are positioned in a substantially linear configuration along a same axis such that the first electrical field and the second electrical field are in an axial bias configuration, and the first electrical field and the second electrical field interfere with each other at an area of overlap to produce a beat signal.

### FIELD

The present disclosure is generally related to electrical stimulation of a subject, more particularly, to an apparatus and method for electrical stimulation using an interferential current pattern for treating certain conditions.

### BACKGROUND

Electrical stimulation of the posterior spinal cord, spinal cord stimulation (SCS), has developed into an effective therapeutic tool for treating chronic pain conditions. However, very little is known about the sites of activation or the neural mechanisms evoked by SCS that relieve pain and promote changes in the function of somatic and visceral structures.

Spinal Cord Stimulation is most commonly used for patients with chronic intractable pain syndromes. It has also been useful for treating movement disorders and is occasionally used following head injuries. However, one complication with SCS is that of accommodation or habituation to the stimulation signal. Accommodation is when the body habituates or becomes accustomed to an activity or signal and then starts to ignore or “tune it out”. By varying the signal or keeping the focal point of the signal moving, accommodation can be minimized.

Dorsal Column Stimulation (DCS) or SCS using an electrical current pattern has shown to be beneficial in treating chronic pain disorders in patients. Traditional SCS stimulation can be limited because of a spread of the stimulating electrical field within cerebral spinal fluid as intensity of stimulation increases. This is due to the highly conductive nature of cerebral spinal fluid (CSF) as compared to the less conductive nature of the spinal cord tissue itself. Frequently, patient satisfaction with electrical stimulation is compromised by the recruitment of adjacent neuronal structures that, when activated, can create discomfort, motor contractions, and outright pain. The efficacy of the therapy is thus limited.

Electrical stimulation has also been shown to be useful to treat certain other conditions. Success of the treatment often is limited to an ability at which the stimulation can be effectively delivered and maintained to a location of pain in the subject.

### SUMMARY

Within examples, a method for electrical stimulation of a subject is described, comprising creating multiple circuits using implantable electrodes positioned in the subject, transmitting a signal of a first frequency through a first circuit of the multiple circuits and the first circuit generates a first electrical field, and transmitting a signal of a second frequency through a second circuit of the multiple circuits and the second circuit generates a second electrical field. The implantable electrodes are positioned in a substantially linear configuration along a same axis such that the first electrical field and the second electrical field are in an axial bias configuration, and the first electrical field and the second electrical field interfere with each other at an area of overlap to produce a beat signal.

In other examples, a method for electrical stimulation of a subject is described, comprising transmitting a signal of a first frequency through a first circuit created between a first pair of implantable electrodes positioned in the subject and the first circuit generates a first electrical field, and transmitting a signal of a second frequency through a second circuit created between a second pair of implantable electrodes positioned in the subject and the second circuit generates a second electrical field. The first pair of implantable electrodes and the second pair of implantable electrodes are positioned in a substantially linear configuration along a same axis such that the first electrical field and the second electrical field are in an axial bias configuration, and the first electrical field and the second electrical field interfere with each other at an area of overlap to produce a beat signal.

In other examples, an electrical stimulator for electrical stimulation of a subject is described, comprising an interferential current generator which generates an interferential alternating current output comprising first signals and second signals, and multiple circuits created using implantable electrodes. The implantable electrodes have a first end and a second end, and the first ends are coupled to the interferential current generator and the second ends are configured to be positioned in the subject, The first signals are transmitted through a first circuit of the multiple circuits to generate a first electrical field, the second signals are transmitted through a second circuit of the multiple circuits to generate a second electrical field, and the implantable electrodes are positioned in a substantially linear configuration along a same axis such that the first electrical field of the first circuit and the second electrical field of the second circuit are in an axial bias configuration. The first electrical field and the second electrical field interfere with each other at an area of overlap to produce a beat signal.

These as well as other aspects, advantages, and alternatives will become apparent to those of ordinary skill in the art by reading the following detailed description, with reference where appropriate to the accompanying drawings.

### BRIEF DESCRIPTION OF THE DRAWINGS

Many aspects of the disclosure can be understood with reference to the following drawings. The components in the drawings are not necessarily to scale. Moreover, in the drawings, like reference numerals designate corresponding parts throughout the several views.

FIG. 1 illustrates an example of an electrical stimulator for electrical stimulation of a subject, according to an example implementation.

FIG. 2 illustrates example quadripolar leads on which the implantable electrodes are provided, according to an example implementation.

FIG. 3 illustrates a single lead with multiple electrodes in which multiple circuits are created to generate electrical fields in an axial bias configuration for electrical stimulation, according to an example implementation.

FIG. 4 illustrates the single lead with multiple electrodes in which multiple circuits are created to generate electrical fields in an axial bias configuration for electrical stimulation, according to another example implementation.

FIG. 5 illustrates the single lead with multiple electrodes in which multiple circuits are created to generate electrical fields in an axial bias configuration for electrical stimulation, according to another example implementation.

FIG. 6 illustrates the single lead with multiple electrodes in which multiple circuits are created to generate electrical fields in an axial bias configuration for electrical stimulation, according to another example implementation.

FIG. 7 illustrates the single lead with multiple electrodes in which multiple circuits are created to generate electrical fields in an axial bias configuration for electrical stimulation, according to another example implementation.

FIG. 8 illustrates the single lead with multiple electrodes in which multiple circuits are created to generate electrical fields in an axial bias configuration for electrical stimulation, according to another example implementation.

FIG. 9 illustrates the single lead with multiple electrodes in which multiple circuits are created to generate electrical fields in an axial bias configuration for electrical stimulation, according to another example implementation.

FIG. 10 illustrates a dual lead arrangement with multiple electrodes in which multiple circuits are created to generate electrical fields in an axial bias configuration for electrical stimulation, according to an example implementation.

FIG. 11 illustrates a dual lead arrangement with multiple electrodes in which multiple circuits are created to generate electrical fields in an axial bias configuration for electrical stimulation, according to an example implementation.

FIG. 12 includes graphs illustrating sinewave representations of the first signal, the second signal, and a resulting beat signal, according to an example implementation.

FIG. 13 shows a flowchart of an example of a method for electrical stimulation of a subject, according to an example embodiment.

FIG. 14 shows a flowchart of an example of a method for electrical stimulation of a subject, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Examples described herein provide an apparatus and method for electrical stimulation of a subject, such as for many different types of treatment and applications. Within examples, an electrical stimulator is provided that includes implantable electrodes, and interferential stimulation is used to produce a beat frequency signal that is directionally controlled to appropriate targets within the subject. An effective area of stimulation is controlled by the quantity of electrodes, and positioning of the electrodes and electrode interference pattern orientation.

Interferential current provides directional control, decreased accommodation or habituation, and increased depth of penetration in comparison to other standard implantable stimulation systems and accompanying surgical leads. Amplitudes of outputs in respective circuits may be modulated to increase an area of targeted stimulation. Within examples, to target specific areas of the subject using modulation of the circuit outputs, the beat frequency signal would be directionally controlled and/or depths of penetration are controlled.

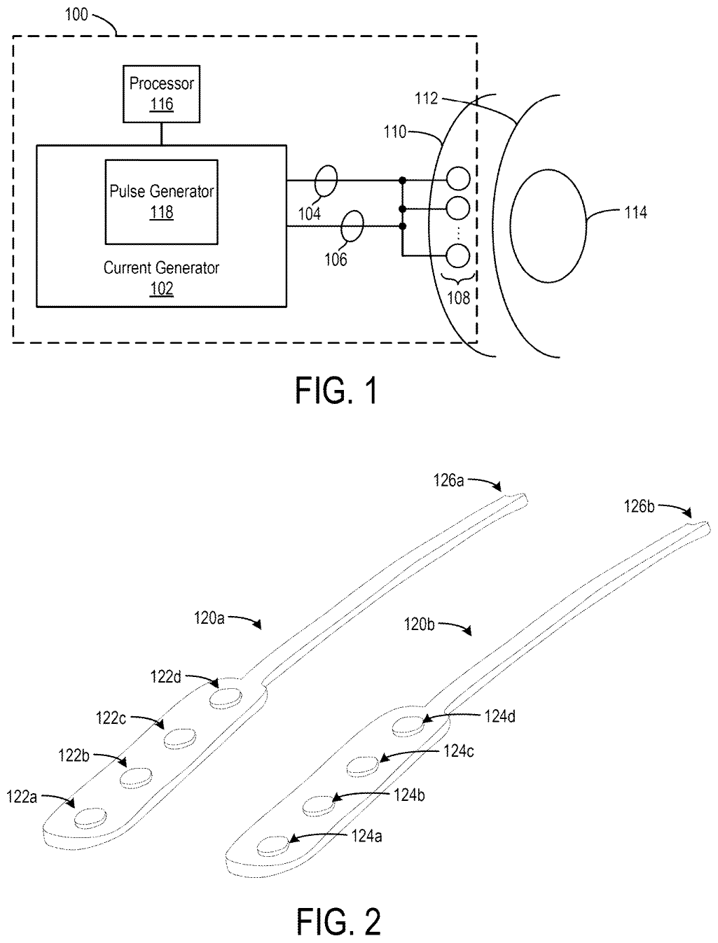

FIG. 1 illustrates an example of an electrical stimulator 100 for electrical stimulation of a subject, according to an example implementation. The electrical stimulator 100 includes an interferential current generator 102 which generates an interferential alternating current output comprising first signals 104 and second signals 106, and multiple circuits created using implantable electrodes 108. The implantable electrodes 108 have a first end and a second end. The first ends are coupled to the interferential current generator 102 and the second ends are configured to be positioned in the subject, such as to tissue 110 of the subject. The first end is connected to the second end through a wire or wire pad. The second end includes a portion of the electrode capable of delivering electrical pulses, and thus, may be an electrode pad. In one example, the implantable electrodes 108 (or portions of the implantable electrodes including the electrode pad) are implanted to a dura matter in an epidural space 112 at predetermined locations proximate to a subject's spinal cord 114. Other example uses are described below.

The electrical stimulator 100 described herein may be fully implanted into a subject, or portions of the electrical stimulator 100 may be implanted and portions remain exterior of the subject. As an example, the implantable electrodes 108 may be implantable, as described, and the interferential current generator 102 and a power source can be external and coupled to the implanted electrodes 108 through wires. In other examples, coupling may occur through a wireless link (e.g., radio frequency (RF) link) from the interferential current generator 102 to the implantable electrodes 108, such that the electrodes are implanted and the interferential current generator 102 is not implanted. The RF carrier frequency can be in the MHz, GHz or THz range and will induce a current in an implanted receiver that is linked or connected to the implantable electrodes 108. The RF carrier frequency can range from about 1 MHz through about 20 THz.

In still other examples, the interferential current generator 102 is implantable in the subject (and a power source connected to the interferential current generator 102 may be implanted as well), and the implantable electrodes 108 are further implanted. The interferential current generator 102 may be implanted near or in the brachial plexus, or near or underneath the 12^{th} rib bone, for example.

In operation of the electrical stimulator 100, the first signals 104 are transmitted through a first circuit of the multiple circuits to generate a first electrical field, the second signals 106 are transmitted through a second circuit of the multiple circuits to generate a second electrical field, and the implantable electrodes 108 are positioned in a substantially linear configuration along a same axis such that the first electrical field of the first circuit and the second electrical field of the second circuit are in an axial bias configuration. The first electrical field and the second electrical field interfere with each other at an area of overlap to produce a beat signal.

In some examples, the electrical stimulator 100 also includes a processor 116 coupled to the interferential current generator 102, and the processor 116 is programmed to cause the interferential current generator 102 to send the first signals 104 and the second signals 106 at selected frequencies, voltage levels, and time periods.

The interferential current generator 102 includes a pulse generator 118 that generates digital signal pulses, and the processor 116 connects to or is in communication with the pulse generator 118 to cause generation of digital signal pulses to approximate a sine-wave-like output waveform. For example, the output may be a sinewave, pseudo sinewave, or some sine-wave-like continuous waveform that are in-phase. In other examples, the output includes a square wave.

The pulse generator 118 generates individual pulses of differing widths and resultant amplitudes. In some examples, the pulse width is set in a range from about 0 to about 2.5 microseconds (ms), from about 2.5 ms to about 5 ms, or from about 5 ms to about 10 ms. When those differing pulses are driven into a transformer (not shown), the pseudo-sine-wave is produced.

The pulse generator 118 also generates a range of outputs, such as amplitudes within a range of about 5 mA to about 9 mA, about 9 mA to about 10 mA, and about 10 mA to about 18 mA, for example, depending on the patient's needs for pain treatment.

The processor 116 may be or include a field-programmable gate array (FPGA) used to shape multiple pulsatile waveforms to approximate the output of a sine-wave generator instead of or in addition to a digital signal processor. The FPGA is an integrated circuit that can be programmed in the field after it is manufactured and allows its user to adjust the circuit output as desired. Thus, in an alternative example, the processor 116 may be replaced with the FPGA. An FPGA device can allow for complex digital signal processing applications such as finite impulse response filters, forward error correction, modulation-demodulation, encryption and applications.

The processor 116 may include internal memory (non-transitory memory as well as buffer/transitory type memory) to store instructions for execution to cause the electrical stimulator 100 to perform functions as described herein. In addition or alternatively, in one example the electrical stimulator 100 includes discrete internal memory to which the processor is in communication (through a traditional bus communication), and the internal memory stores instructions for execution to cause the electrical stimulator 100 to perform functions as described herein.

The electrical stimulator 100 may include further components as well, such as a power source and other circuitry to perform functions described herein.

Within examples, as mentioned above, the processor 116 is in communication with the interferential current generator 102 to cause the interferential current generator 102 to send different signals at different time periods for waveform generation for electrical stimulation treatment.

FIG. 2 illustrates example quadripolar leads 120a-b on which the implantable electrodes 108 are provided, according to an example implementation. FIG. 2 illustrates two quadripolar leads 120a-b, however, more or fewer leads may be used depending upon an electrode placement and arrangement. Each quadripolar lead 120a-b includes multiple implantable electrodes shown as four electrode pads 122a-d and 124a-d. The use of quadripolar leads allows a greater target treatment stimulation area of the subject. However, electrical stimulators of the present disclosure may also apply to use of two bipolar or octapolar lead systems, and other suitable devices with any number of electrode pads included such as four, six, eight, ten, . . . , or up to thirty or thirty-two, for example. The quadripolar leads 120a-b include first ends 126a-b that couple to the current generator 102. The implantable electrodes 108 could be activated in various combinations and patterns, and not just as shown in the drawings.

In operation, the current generator 102 generates an interferential output including the first signals 104 and the second signals 106 having different first and second frequencies. Selected electrodes of the implantable electrodes 108 carry one of the first signals 104 and the second signals 106 to create separate circuits. Where a first circuit (created between two electrodes) and a second circuit (created between two electrodes) interfere, a resultant beat frequency will be a difference between frequencies of the two circuits, and an amplitude will be additive and greater than either circuit alone. Within other examples, the resultant beat frequency signal may have a frequency within a range of more than 250 Hz to about 15,000 Hz.

Within many examples described below, multiple circuits are created between the implantable electrodes 108 in a number of ways. For example, multiple circuits can be created using three electrodes, where one electrode is common among two circuits. In other examples, multiple circuits can be created using four electrodes, so that separate circuits are created between separate pairs of electrodes. Still further, multiple circuits can be created using electrodes on a single lead, or multiple circuits can be created using two separate leads positioned in the substantially linear configuration approximately end-to-end, and a distance, measured perpendicular to the same axis, between the first lead and the second lead is less than about 2 mm.

Thus, in some examples, the implantable electrodes 108 are included on a single lead, and the implantable electrodes 108 are independently controllable to be arranged as positive and negative electrode pairs for creation of the first circuit and the second circuit using a single lead. Further, using a single lead, multiple circuits can be created so that a first circuit is between a first implantable electrode and a second implantable electrode and a second circuit is between the first implantable electrode and a third implantable electrode, and the first circuit and the second circuit have a common implantable electrode. In other examples, using a single lead, multiple circuits can be created so that a first circuit is between a first implantable electrode and a second implantable electrode and a second circuit is between a third implantable electrode and a fourth implantable electrode.

In still other examples, the implantable electrodes include a first pair of implantable electrodes and a second pair of implantable electrodes positioned in the substantially linear configuration along the same axis.

In summary, the multiple circuits are created in many ways including using three electrodes on a single lead, using four electrodes on a single lead, using more than four electrodes on a single lead (such as for more than two circuits), using three electrodes from two different leads, using four electrodes from two different leads, or using more than four electrodes from two different leads. Examples are described and shown in Figures below.

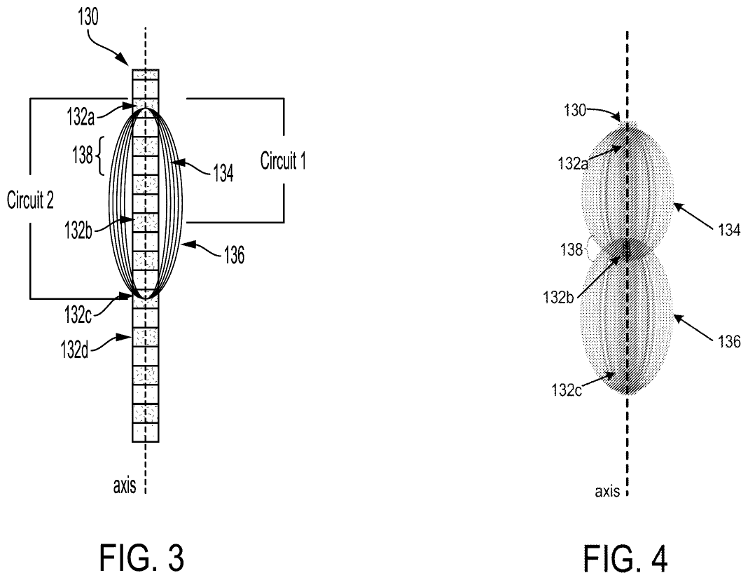

FIG. 3 illustrates a single lead 130 with multiple electrodes in which multiple circuits are created to generate electrical fields in an axial bias configuration for electrical stimulation, according to an example implementation. The lead 130 includes implantable electrodes 132a-d, and in one example, the lead 130 may take a form of one of the leads 120a-b shown in FIG. 2, for example (although, the lead 130 is shown to include further unlabeled electrodes as well).

Each of the implantable electrodes 132a-d is independently controllable to be operated as a cathode or anode, and any combination of the implantable electrodes 132a-d can be selected to create one or more circuits. For physiological purposes, a nerve has a negative internal charge and is polarized at rest so as to be ready to fire, and the electrons (negative internal charge) attract positive charge on an outside of the nerve resulting in depolarization. Thus, physiologically, a cathode is considered a negative contact, and an anode is where the negative charge accumulates so that the anode is considered a positive contact that attracts negative charge.

In FIG. 3, a first circuit is created between the implantable electrodes 132a and 132b by arranging the implantable electrode 132a as a cathode or negative contact and by arranging the implantable electrode 132b as an anode or positive contact. A second circuit is created between the implantable electrodes 132a and 132c by arranging the implantable electrode 132c as another anode or positive contact. In this example, the first circuit and the second circuit share the implantable electrode 132a as the anode, and the implantable electrode 132d is not used.

In operation of the arrangement shown in FIG. 3, three electrodes of the lead 130 are used to create two separate circuits. Following, the interferential current generator 102 is operated to transmit a signal of a first frequency through the first circuit to generate a first electrical field 134, to transmit a signal of a second frequency through the second circuit to generate a second electrical field 136, and the first electrical field 134 and the second electrical field 134 interfere with each other at an area of overlap 138 to produce a beat signal. Where the first electrical field 134 and the second electrical field 136 superimpose or overlap, the resultant beat signal will be the difference between the frequencies of the two circuits and the amplitude will be additive and greater than either circuit alone.

In FIG. 3, the implantable electrodes 132a, 132b, and 132c are positioned in a substantially linear configuration along a same axis such that the first electrical field 134 and the second electrical field 136 are in an axial bias configuration.

FIG. 4 illustrates the single lead 130 with multiple electrodes in which multiple circuits are created to generate electrical fields in an axial bias configuration for electrical stimulation, according to another example implementation. In FIG. 4, the first circuit is created between the implantable electrodes 132a and 132b by arranging the implantable electrode 132a as an anode or positive contact and by arranging the implantable electrode 132b as the cathode or negative contact. The second circuit is created between the implantable electrodes 132b and 132c by arranging the implantable electrode 132c as another anode or positive contact. In this example, the first circuit and the second circuit share the implantable electrode 132b as the cathode.

In operation of the arrangement shown in FIG. 4, three electrodes of the lead 130 are used to create two separate circuits. Following, the interferential current generator 102 is operated to transmit a signal of a first frequency through the first circuit to generate a first electrical field 134, to transmit a signal of a second frequency through the second circuit to generate a second electrical field 136, and the first electrical field 134 and the second electrical field 134 interfere with each other at an area of overlap 138 to produce a beat signal. Where the first electrical field 134 and the second electrical field 136 superimpose or overlap, the resultant beat signal will be the difference between the frequencies of the two circuits and the amplitude will be additive and greater than either circuit alone.

Similar to the arrangement in FIG. 3, within FIG. 4, the implantable electrodes 132a, 132b, and 132c are positioned in a substantially linear configuration along a same axis such that the first electrical field 134 and the second electrical field 136 are in an axial bias configuration.

Thus, as shown by the examples in FIGS. 3 and 4, the single lead 130 includes a plurality of electrodes arranged in a linear electrode array along the axis, and the altering selection of usage of the electrodes along the single lead alters a longitudinal positioning of the beat signal (or area of overlap 138). Similarly, the longitudinal positioning of the beat signal can be altered by changing a configuration of the first circuit and the second circuit to be operated from among the plurality of electrodes of the linear electrode array or to be operated differently from among the plurality of electrodes of the linear electrode array. For example, changing the shared or common electrode 132b to be an anode in FIG. 3 to a cathode in FIG. 4 changes how the electrical fields interfere for creation of the beat signal.

The interferential current generator 120 is operated in many ways to transmit the first signals 104 and the second signals 106 of different frequencies. In one example, transmitting the signal of the first frequency across the first circuit includes transmitting the signal at a frequency between about 1,000 Hz to about 20,000 Hz, and transmitting the signal of the second frequency across the second circuit includes transmitting the signal at a frequency between about 1,000 Hz to about 20,000 Hz. For generation of the beat signal, the first frequency is different from the second frequency.

Frequencies of signals may be transmitted through the first circuit and the second circuit within ranges of about 0 to about 20,000 Hz, or any ranges than can result in the beat signal having a frequency in a range of more than 0 Hz to about 5,000 Hz, for example. The beat signal frequency results from interference of the two signals from the first circuit and the second circuit (e.g., for a frequency of 10,000 Hz at the first circuit creating a first electrical field interfering with a second electrical field generated by the second circuit due to a frequency of 12,000 Hz results in a beat signal frequency of about 2,000 Hz).

Based on combinations of the frequencies used and transmitted in the first circuit and the second circuit, the beat signal may be in a range of more than 0 Hz to about 5,000 Hz. Thus, within examples, signals are transmitted in a range of frequencies between about 12,000 Hz to about 15,000 Hz, a range of frequencies between about 13,000 Hz to about 15,000 Hz, a range of frequencies between about 14,000 Hz to about 15,000 Hz, a range of frequencies between about 10,000 Hz to about 15,000 Hz, a range of frequencies between about 6,000 Hz to about 9,000 Hz, a range of frequencies between about 7,000 Hz to about 9,000 Hz, a range of frequencies between about 8,000 Hz to about 9,000 Hz, a range of frequencies between about 9,000 Hz to about 12,000 Hz, a range of frequencies between about 10,000 Hz to about 12,000 Hz, a range of frequencies between about 11,000 Hz to about 13,000 Hz, a range of frequencies between about 13,000 Hz to about 15,000 Hz, a range of frequencies between about 3,000 Hz to about 5,000 Hz, a range of frequencies between about 3,000 Hz to about 7,000 Hz, a range of frequencies between about 3,000 Hz to about 6,000 Hz, a range of frequencies between about 5,000 Hz to about 8,000 Hz, a range of frequencies between about 1,000 Hz to about 5,000 Hz, or any other ranges between 1,000 Hz to about 20,000 Hz.

Thus, any signals in a range of frequency between about 1,000 Hz and 20,000 Hz can be used and transmitted to the first circuit and the second circuit to caused interference of electrical fields resulting in a beat signal generated that is in a range of about 0-5,000 beats per second (BPS).

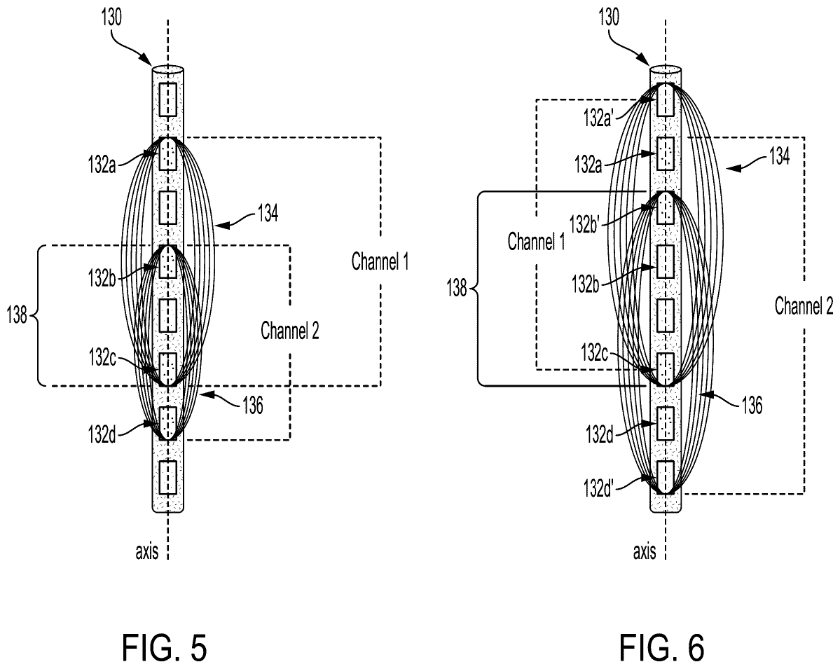

FIG. 5 illustrates the single lead 130 with multiple electrodes in which multiple circuits are created to generate electrical fields in an axial bias configuration for electrical stimulation, according to another example implementation. In FIG. 5, the first circuit is created between the implantable electrodes 132a and 132c by arranging the implantable electrode 132a as a cathode or negative contact and by arranging the implantable electrode 132c as the anode or positive contact. The second circuit is created between the implantable electrodes 132b and 132d by arranging the implantable electrode 132b as the anode or positive contact and by arranging the implantable electrode 132d as the cathode or negative contact. In this example, the first circuit and the second circuit do not share any implantable electrodes, and each of the first circuit and the second circuit are created using a different pair of implantable electrodes.

In operation of the arrangement shown in FIG. 5, four electrodes of the lead 130 are used to create two separate circuits. Following, the interferential current generator 102 is operated to transmit a signal of a first frequency through the first circuit to generate a first electrical field 134, to transmit a signal of a second frequency through the second circuit to generate a second electrical field 136, and the first electrical field 134 and the second electrical field 134 interfere with each other at an area of overlap 138 to produce a beat signal. Where the first electrical field 134 and the second electrical field 136 superimpose or overlap, the resultant beat signal will be the difference between the frequencies of the two circuits and the amplitude will be additive and greater than either circuit alone.

In FIG. 5, the implantable electrodes 132a, 132b, 132c, and 132d are positioned in a substantially linear configuration along a same axis such that the first electrical field 134 and the second electrical field 136 are in an axial bias configuration.

FIG. 6 illustrates the single lead 130 with multiple electrodes in which multiple circuits are created to generate electrical fields in an axial bias configuration for electrical stimulation, according to another example implementation. In FIG. 6, the first circuit is created between the implantable electrodes 132a′ and 132c by arranging the implantable electrode 132a′ as the anode or positive contact and by arranging the implantable electrode 132c as the cathode or negative contact. The second circuit is created between the implantable electrodes 132b′ and 132d′ by arranging the implantable electrode 132b′ as the cathode or negative contact and by arranging the implantable electrode 132d′ as the anode or positive contact. In this example, the first circuit and the second circuit do not share any implantable electrodes, and each of the first circuit and the second circuit are created using a different pair of implantable electrodes. In addition, in FIG. 6, the implantable electrodes 132a, 132b, and 132d are not operated.

In operation of the arrangement shown in FIG. 6, four electrodes of the lead 130 are used to create two separate circuits. Following, the interferential current generator 102 is operated to transmit a signal of a first frequency through the first circuit to generate a first electrical field 134, to transmit a signal of a second frequency through the second circuit to generate a second electrical field 136, and the first electrical field 134 and the second electrical field 134 interfere with each other at an area of overlap 138 to produce a beat signal. Where the first electrical field 134 and the second electrical field 136 superimpose or overlap, the resultant beat signal will be the difference between the frequencies of the two circuits and the amplitude will be additive and greater than either circuit alone.

In FIG. 6, the implantable electrodes 132a′, 132b′, 132c, and 132d′ are positioned in a substantially linear configuration along a same axis such that the first electrical field 134 and the second electrical field 136 are in an axial bias configuration.

Within the arrangements shown in FIGS. 5 and 6 the first circuit is created between a first pair of implantable electrodes positioned in the subject and the second circuit is created between a second pair of implantable electrodes positioned in the subject. Each of the first pair of implantable electrodes and the second pair of implantable electrodes are positioned in a substantially linear configuration along a same axis such that the first electrical field and the second electrical field are in an axial bias configuration. In addition, the first pair of implantable electrodes and the second pair of implantable electrodes are included on the single lead 130. As shown, altering selection of a first electrode of the first pair of implantable electrodes on the single lead 130 alters a longitudinal positioning of the beat signal.

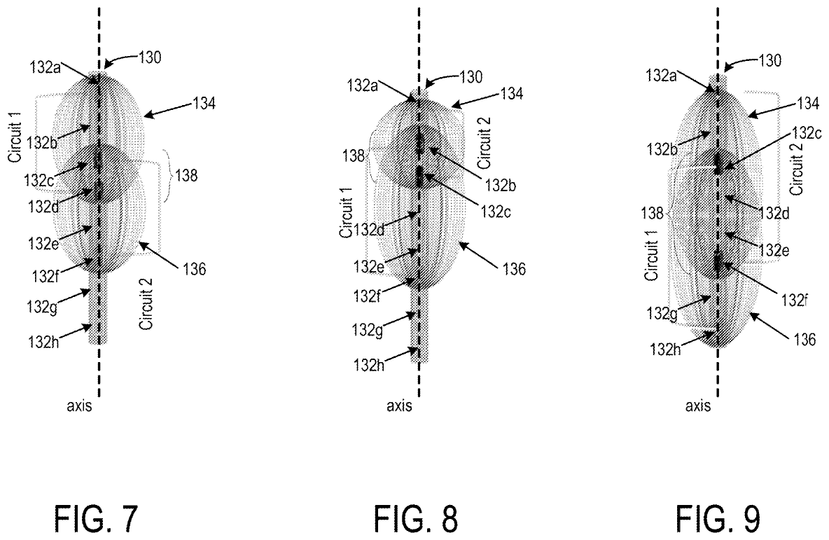

FIGS. 7-9 illustrate further examples of using the single lead 130 with four electrodes operated in different configurations to create multiple circuits and a resultant beat signal at different locations.

FIG. 7 illustrates the single lead 130 with multiple electrodes in which multiple circuits are created to generate electrical fields in an axial bias configuration for electrical stimulation, according to another example implementation. In FIG. 7, the single lead 130 is shown to include eight electrodes labeled as 132a-h. The first circuit is created between a pair of the implantable electrodes 132a and 132d by arranging the implantable electrode 132a as the anode or positive contact and by arranging the implantable electrode 132d as the cathode or negative contact. The second circuit is created between a pair of the implantable electrodes 132c and 132f by arranging the implantable electrode 132c as the cathode or negative contact and by arranging the implantable electrode 132f as the anode or positive contact. In this example, the first circuit and the second circuit do not share any implantable electrodes, and each of the first circuit and the second circuit are created using a different pair of implantable electrodes. In addition, in FIG. 7, the implantable electrodes 132b, 132e, 132g, and 132h are not operated.

In operation of the arrangement shown in FIG. 7, four electrodes of the lead 130 are used to create two separate circuits. Following, the interferential current generator 102 is operated to transmit a signal of a first frequency through the first circuit to generate a first electrical field 134, to transmit a signal of a second frequency through the second circuit to generate a second electrical field 136, and the first electrical field 134 and the second electrical field 134 interfere with each other at an area of overlap 138 to produce a beat signal.

FIG. 8 illustrates the single lead 130 with multiple electrodes in which multiple circuits are created to generate electrical fields in an axial bias configuration for electrical stimulation, according to another example implementation. In FIG. 8, the first circuit is created between a pair of the implantable electrodes 132b and 132f by arranging the implantable electrode 132b as the cathode or negative contact and by arranging the implantable electrode 132f as the anode or positive contact. The second circuit is created between a pair of the implantable electrodes 132a and 132c by arranging the implantable electrode 132a as the anode or positive contact and by arranging the implantable electrode 132c as the cathode or negative contact. In this example, the first circuit and the second circuit do not share any implantable electrodes, and each of the first circuit and the second circuit are created using a different pair of implantable electrodes. In addition, in FIG. 8, the implantable electrodes 132d, 132e, 132g, and 132h are not operated.

In operation of the arrangement shown in FIG. 8, four electrodes of the lead 130 are used to create two separate circuits. Following, the interferential current generator 102 is operated to transmit a signal of a first frequency through the first circuit to generate a first electrical field 134, to transmit a signal of a second frequency through the second circuit to generate a second electrical field 136, and the first electrical field 134 and the second electrical field 136 interfere with each other at an area of overlap 138 to produce a beat signal.

FIG. 9 illustrates the single lead 130 with multiple electrodes in which multiple circuits are created to generate electrical fields in an axial bias configuration for electrical stimulation, according to another example implementation. In FIG. 9, the first circuit is created between a pair of the implantable electrodes 132c and 132h by arranging the implantable electrode 132c as the cathode or negative contact and by arranging the implantable electrode 132h as the anode or positive contact. The second circuit is created between a pair of the implantable electrodes 132a and 132f by arranging the implantable electrode 132a as the anode or positive contact and by arranging the implantable electrode 132f as the cathode or negative contact. In this example, the first circuit and the second circuit do not share any implantable electrodes, and each of the first circuit and the second circuit are created using a different pair of implantable electrodes. In addition, in FIG. 9, the implantable electrodes 132b, 132d, 132e, and 132g are not operated.

In operation of the arrangement shown in FIG. 9, four electrodes of the lead 130 are used to create two separate circuits. Following, the interferential current generator 102 is operated to transmit a signal of a first frequency through the first circuit to generate a first electrical field 134, to transmit a signal of a second frequency through the second circuit to generate a second electrical field 136, and the first electrical field 134 and the second electrical field 136 interfere with each other at an area of overlap 138 to produce a beat signal.

In each of FIGS. 7-9, the implantable electrodes are all positioned in a substantially linear configuration along a same axis such that the first electrical field 134 and the second electrical field 136 are in an axial bias configuration. As shown in FIGS. 7-9, by changing selection of electrodes for use in creating the multiple circuits, the location of the beat signal can be moved longitudinally along the axis. In addition, a shape or focus of the beat signal can be altered as well to be more narrow (as shown in FIG. 7) to more elongated (as shown in FIG. 9).

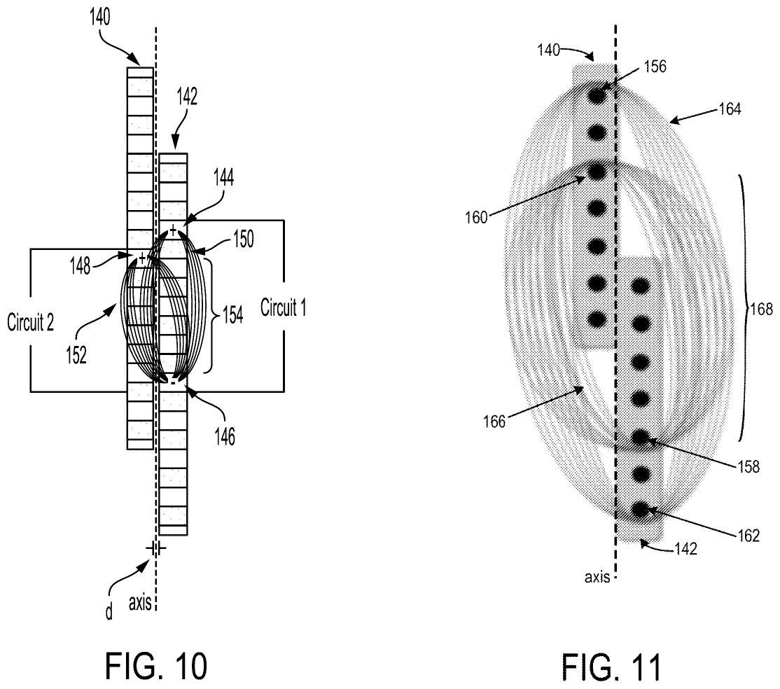

FIG. 10 illustrates a dual lead arrangement with multiple electrodes in which multiple circuits are created to generate electrical fields in an axial bias configuration for electrical stimulation, according to an example implementation. In FIG. 10, a first lead 140 and a second lead 142 each include implantable electrodes. The two circuits are created using three electrodes, where each circuit shares a common cathode or negative contact. For example, a first circuit is created between an implantable electrode 144 (arranged as an anode or positive contact) on the lead 142 and an implantable electrode 146 (arranged as a cathode or negative contact) on the lead 142. A second circuit is created between an implantable electrode 148 (arranged as an anode or positive contact) on the lead 140 and the implantable electrode 146 (arranged as a cathode or negative contact) on the lead 142.

In operation of the arrangement shown in FIG. 10, three electrodes (one on the lead 140 and two on the lead 142) are used to create two separate circuits. Following, the interferential current generator 102 is operated to transmit a signal of a first frequency through the first circuit to generate a first electrical field 150, to transmit a signal of a second frequency through the second circuit to generate a second electrical field 152, and the first electrical field 150 and the second electrical field 152 interfere with each other at an area of overlap 154 to produce a beat signal.

In FIG. 10, the first lead 140 and the second lead 142 are positioned in the substantially linear configuration along the same axis, and a distance, measured perpendicular to the same axis, between the first lead 140 and the second lead 142 is less than about 2 mm. In FIG. 10, the first lead 140 and the second lead 142 are shown immediately adjacent each other so that the distance, measured perpendicular to the same axis, between the first lead 140 and the second lead 142 is effectively zero mm.

In other examples, a dual lead arrangement is implemented so that the first lead 140 and the second lead 142 are positioned in the substantially linear configuration approximately end-to-end, and a distance (d), measured perpendicular to the same axis, between the first lead 140 and the second lead 142 is less than about 2 mm. In other examples, the distance (d) is less than about 1 mm, less than about 1 mm-2 mm, or less than about 0.5-1 mm.

FIG. 11 illustrates a dual lead arrangement with multiple electrodes in which multiple circuits are created to generate electrical fields in an axial bias configuration for electrical stimulation, according to an example implementation. In FIG. 11, the first lead 140 and the second lead 142 each include implantable electrodes. The two circuits are created using four electrodes, where each circuit uses a separate pair of electrodes with one electrode on the first lead 140 and one electrode on the second lead 142. For example, the first circuit is created between an implantable electrode 156 (arranged as an anode or positive contact) on the lead 140 and an implantable electrode 158 (arranged as a cathode or negative contact) on the lead 142. A second circuit is created between an implantable electrode 160 (arranged as an anode or positive contact) on the lead 140 and the implantable electrode 162 (arranged as a cathode or negative contact) on the lead 142.

In operation of the arrangement shown in FIG. 11, four electrodes (two on the lead 140 and two on the lead 142) are used to create two separate circuits. Following, the interferential current generator 102 is operated to transmit a signal of a first frequency through the first circuit to generate a first electrical field 164, to transmit a signal of a second frequency through the second circuit to generate a second electrical field 166, and the first electrical field 164 and the second electrical field 166 interfere with each other at an area of overlap 168 to produce a beat signal.

In FIG. 11, the first lead 140 and the second lead 142 are positioned in the substantially linear configuration along the same axis, and a distance, measured perpendicular to the same axis, between the first lead 140 and the second lead 142 is less than about 2 mm. In FIG. 11, the first lead 140 and the second lead 142 are shown immediately adjacent each other so that the first lead 140 and the second lead 142 are in a substantially linear configuration and arranged approximately end-to-end so that at least one electrode on the first lead 140 is positioned adjacent to at least one electrode on the second lead 142.

In some examples, the first circuit is created using the implantable electrodes on the first lead 140, and the second circuit is created using the implantable electrodes on the second lead 142. By positioning the first lead 140 and the second lead 142 along the same axis as shown in FIG. 11, an axial bias configuration can be established.

Thus, within examples described herein, an axial bias configuration can be implemented to generate a beat signal useful for electrical stimulation. The axial bias configuration can be established using three electrodes on a single lead, using four electrodes on a single lead, using three electrodes from two different leads, or using four electrodes from two different leads, for example.

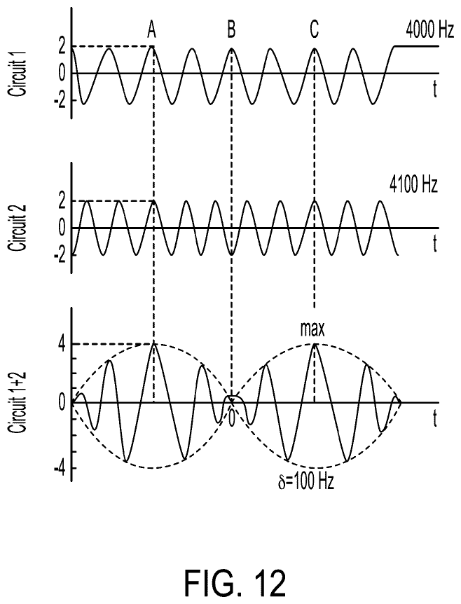

FIG. 12 includes graphs illustrating sinewave representations of the first signal, the second signal, and a resulting beat signal, according to an example implementation. In FIG. 12, a first signal having a first frequency of 4,000 Hz is transmitted across the first circuit (which may be any of the first circuits shown and described with reference to FIGS. 3-11), and a second signal having a second frequency of 4,100 Hz is transmitted across the second circuit (which may be any of the second circuits shown and described with reference to FIGS. 3-11). Interference of electrical fields generated by the transmission of the first and second signals causes a beat signal to be generated. As waves of two different frequencies interfere, the waves are either constructive (additive amplitudes) or destructive (subtractive amplitudes). A well-defined beat will occur when the amplitudes are identical. A frequency of the beat signal is a difference between the two carrier frequencies. For the example shown in FIG. 12, the beat frequency is 100 Hz.

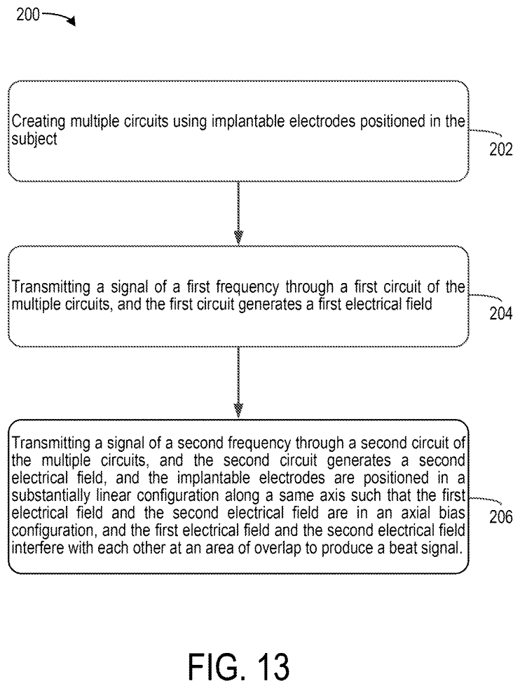

FIG. 13 shows a flowchart of an example of a method 200 for electrical stimulation of a subject, according to an example embodiment. The method shown in FIG. 13 presents an example of a method that, for example, could be used by the stimulator 100 shown in FIG. 1, for example, and may be performed by components of the stimulator 100 in FIG. 1. In some instances, components of the stimulator 100 may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. The method may include one or more operations, functions, or actions as illustrated by one or more of blocks 202-206. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In still further examples, functions of methods described herein are performed by circuitry (processor) executing instructions stored on non-transitory computer-readable medium to cause the electrical stimulator to provide stimulation treatment.

It should be understood that for this and other processes and methods disclosed herein, flowcharts show functionality and operation of one possible implementation of present embodiments. Alternative implementations are included within the scope of the example embodiments of the present disclosure in which functions may be executed out of order from that shown or discussed, including substantially concurrent or in reverse order, depending on the functionality involved, as would be understood by those reasonably skilled in the art.

At block 202, the method 200 includes creating multiple circuits using implantable electrodes positioned in the subject.

In some examples, creating multiple circuits using implantable electrodes positioned in the subject comprises creating the first circuit between a first implantable electrode and a second implantable electrode, and creating the second circuit between the first implantable electrode and a third implantable electrode. In these examples, the first circuit and the second circuit have a common implantable electrode.

In some examples, creating multiple circuits using implantable electrodes positioned in the subject comprises creating the first circuit between a first implantable electrode and a second implantable electrode, and creating the second circuit between a third implantable electrode and a fourth implantable electrode.

In some examples, creating multiple circuits using implantable electrodes positioned in the subject comprises creating the first circuit and the second circuit using the implantable electrodes on a single lead. The single lead includes a plurality of electrodes arranged in a linear electrode array, and the method 200 optionally includes altering selection of a first electrode of the first circuit on the single lead to alter a longitudinal positioning of the beat signal. In addition, the method 200 optionally includes altering a longitudinal positioning of the beat signal by changing a configuration of the first circuit and the second circuit to be operated from among the plurality of electrodes of the linear electrode array.

In some examples, creating multiple circuits using implantable electrodes positioned in the subject comprises creating the first circuit using the implantable electrodes on the first lead, and creating the second circuit using the implantable electrodes on the second lead.

At block 204, the method 200 includes transmitting a signal of a first frequency through a first circuit of the multiple circuits, and the first circuit generates a first electrical field.

At block 206, the method 200 includes transmitting a signal of a second frequency through a second circuit of the multiple circuits, and the second circuit generates a second electrical field. The implantable electrodes are positioned in a substantially linear configuration along a same axis such that the first electrical field and the second electrical field are in an axial bias configuration, and the first electrical field and the second electrical field interfere with each other at an area of overlap to produce a beat signal.

In one example, the first lead and the second lead are positioned in the substantially linear configuration approximately end-to-end, wherein a distance, measured perpendicular to the same axis, between the first lead and the second lead is less than about 2 mm.

In one example, the implantable electrodes are independently controllable to be arranged as positive and negative electrode pairs for creation of the first circuit and the second circuit.

In some examples, the method 200 includes transmitting the signal of the first frequency comprises transmitting the signal at a frequency between about 1,000 Hz to about 20,000 Hz, and transmitting the signal of the second frequency comprises transmitting the signal at a frequency between about 1,000 Hz to about 20,000 Hz, wherein the first frequency is different from the second frequency. In some examples, the beat signal has a frequency within a range of more than 0 Hz to about 5,000 Hz.

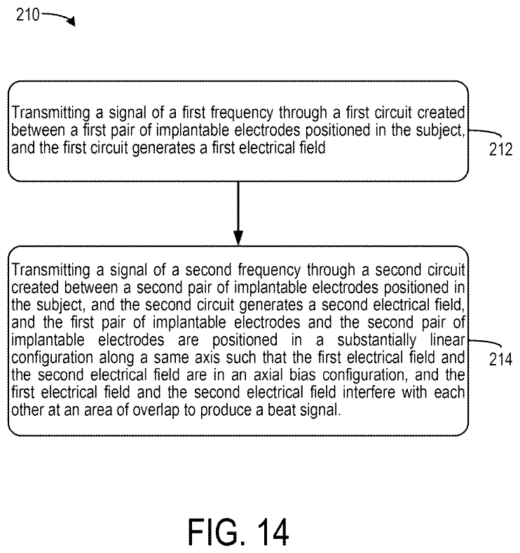

FIG. 14 shows a flowchart of an example of a method 210 for electrical stimulation of a subject, according to an example embodiment. The method shown in FIG. 14 presents an example of a method that, for example, could be used by the stimulator 100 shown in FIG. 1, for example, and may be performed by components of the stimulator 100 in FIG. 1. In some instances, components of the stimulator 100 may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. The method may include one or more operations, functions, or actions as illustrated by one or more of blocks 212-214. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present embodiments. Alternative implementations are included within the scope of the example embodiments of the present disclosure in which functions may be executed out of order from that shown or discussed, including substantially concurrent or in reverse order, depending on the functionality involved, as would be understood by those reasonably skilled in the art.

At block 212, the method 210 includes transmitting a signal of a first frequency through a first circuit created between a first pair of implantable electrodes positioned in the subject, and the first circuit generates a first electrical field.

At block 214, the method 210 includes transmitting a signal of a second frequency through a second circuit created between a second pair of implantable electrodes positioned in the subject, and the second circuit generates a second electrical field.

In the method 210, the first pair of implantable electrodes and the second pair of implantable electrodes are positioned in a substantially linear configuration along a same axis such that the first electrical field and the second electrical field are in an axial bias configuration, and the first electrical field and the second electrical field interfere with each other at an area of overlap to produce a beat signal.

In some examples, the first pair of implantable electrodes and the second pair of implantable electrodes are aligned vertically along a longitudinal axis of the spinal cord to form the first circuit and the second circuit and the first circuit is positioned on the same axis as the second circuit.

In some examples, the first pair of implantable electrodes and the second pair of implantable electrodes are included on a single lead, and the first pair of implantable electrodes and the second pair of implantable electrodes are independently controllable to be arranged as positive and negative electrode pairs for creation of the first circuit and the second circuit. In some examples, the single lead includes a plurality of electrodes arranged in a linear electrode array, and the method 210 further comprises altering selection of a first electrode of the first pair of implantable electrodes on the single lead to alter a longitudinal positioning of the beat signal.

In some examples, the first pair of implantable electrodes are included on a first lead and the second pair of implantable electrodes are included on a second lead, and the first lead and the second lead are positioned in the substantially linear configuration approximately end-to-end, such that a distance (d), measured perpendicular to the same axis, between the first lead and the second lead is less than about 2 mm.

In some examples, the substantially linear configuration approximately end-to-end comprises at least one electrode on the first lead positioned adjacent to at least one electrode on the second lead.

In some examples, the method 210 includes transmitting the signal of the first frequency comprises transmitting the signal at a frequency between about 1,000 Hz to about 20,000 Hz, and transmitting the signal of the second frequency comprises transmitting the signal at a frequency between about 1,000 Hz to about 20,000 Hz, wherein the first frequency is different from the second frequency. In some examples, the beat signal has a frequency within a range of more than 0 Hz to about 5,000 Hz.

The electrical stimulation described herein may be used for many different types of treatment or applications. In one example, the methods described herein include positioning the first pair of implantable electrodes to a dura matter in an epidural space proximate to a spinal cord of the subject, and positioning the second pair of implantable electrodes to the dura matter in the epidural space proximate to the spinal cord of the subject in order to provide spinal cord stimulation treatment. In such examples, the first pair of implantable electrodes and the second pair of implantable electrodes are aligned vertically along a longitudinal axis of the spinal cord to form the first circuit and the second circuit and the first circuit is positioned on the same axis as the second circuit.

Other example applications exist as well and the implantable electrodes may be positioned accordingly, such as proximal to the spinal cord and supportive tissues (Glia and Microglia, interstitial tissue, etc.), transforaminal stimulation of the spinal nerve(s) and spinal nerve root(s) separately or simultaneously and supportive tissues, vertebrae nerves and supportive tissues, peripheral nerves and supportive tissues, and Vagus Nerves and supportive tissues. Still other example applications including for treatment and application to sympathetic and parasympathetic nerves outside the spinal canal may be implemented by positioning the implantable electrodes proximal to paravertebral locations, such as cervical including superior cervical ganglion, the middle cervical ganglion, the cervicothoracic ganglion (stellate ganglion), thoracic, and lumbar or prevertebral locations, such as coeliac, superior mesenteric, inferior mesenteric, and ganglion impar.

Example indications and intended uses of the electrical stimulation include pain treatment (both chronic and acute), blood pressure modulation, blood sugar level modulation (diabetes modulation), inflammation, heart rate and cardiac neuromodulation, neuromodulation of breathing, neuromodulation of other vegetative functions (sympathetic and parasympathetic systems), and anxiety.

As such, within example methods described herein, the methods optionally include positioning the implantable electrodes to space proximate to nervous tissue of the subject, positioning the implantable electrodes to space proximate to vertebral nerves of the subject, positioning the implantable electrodes to space proximate to a dorsal root ganglia of the subject, positioning the first pair of implantable electrodes and positioning the second pair of implantable electrodes to space proximate to Vagus nerves of the subject, or positioning the implantable electrodes to space proximate sympathetic and parasympathetic nerves.

Within yet further examples, system and methods described herein are useful for operation of the electrical stimulator as well as for programming operation of the electrical stimulator 100. To program the electrical stimulator 100 and select electrodes from among the implantable electrodes 108 for use, circuits are created (as described with references to the methods in FIG. 13 or 14), and the subject can indicate where stimulation is physically experienced or felt on their body. As such, the stimulation can be moved left/right or up/down by changing selection of the electrodes from among the implantable electrodes 108 so as to provide the stimulation to a desired area.

By the term “about” and/or the term “substantially” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

It should be emphasized that the above-described embodiments of the present disclosure are merely possible examples of implementations set forth for a clear understanding. Many variations and modifications may be made to the above-described embodiment(s) without departing substantially from the principles of the disclosure. All such modifications and variations are intended to be included herein within the scope of this disclosure.

What is claimed is:

1. A method for electrical stimulation of a subject, the method comprising:
creating multiple circuits using implantable electrodes positioned in the subject;
transmitting a signal of a first frequency through a first circuit of the multiple circuits, wherein the first circuit generates a first electrical field;
transmitting a signal of a second frequency through a second circuit of the multiple circuits, wherein the second circuit generates a second electrical field,
wherein the implantable electrodes are positioned in a substantially linear configuration along a same axis such that the first electrical field and the second electrical field are in an axial bias configuration, and
wherein the first electrical field and the second electrical field interfere with each other at an area of overlap to produce a beat signal.

2. The method of claim 1, wherein creating multiple circuits using implantable electrodes positioned in the subject comprises:
creating the first circuit between a first implantable electrode and a second implantable electrode; and
creating the second circuit between the first implantable electrode and a third implantable electrode,
wherein the first circuit and the second circuit have a common implantable electrode.

3. The method of claim 1, wherein creating multiple circuits using implantable electrodes positioned in the subject comprises:
creating the first circuit between a first implantable electrode and a second implantable electrode; and
creating the second circuit between a third implantable electrode and a fourth implantable electrode.

4. The method of claim 1, wherein creating multiple circuits using implantable electrodes positioned in the subject comprises:
creating the first circuit and the second circuit using the implantable electrodes on a single lead.

5. The method of claim 4, wherein the single lead includes a plurality of electrodes arranged in a linear electrode array, and the method further comprises:
altering selection of a first electrode of the first circuit on the single lead to alter a longitudinal positioning of the beat signal.

6. The method of claim 4, wherein the single lead includes a plurality of electrodes arranged in a linear electrode array, and the method further comprises:
altering a longitudinal positioning of the beat signal by changing a configuration of the first circuit and the second circuit to be operated from among the plurality of electrodes of the linear electrode array.

7. The method of claim 1, wherein the implantable electrodes are provided on a first lead and a second lead, and wherein creating multiple circuits using implantable electrodes positioned in the subject comprises:
creating the first circuit using the implantable electrodes on the first lead; and
creating the second circuit using the implantable electrodes on the second lead.

8. The method of claim 7, wherein the first lead and the second lead are positioned in the substantially linear configuration approximately end-to-end, wherein a distance, measured perpendicular to the same axis, between the first lead and the second lead is less than about 2 mm.

9. The method of claim 1, wherein the implantable electrodes are independently controllable to be arranged as positive and negative electrode pairs for creation of the first circuit and the second circuit.

10. The method of claim 1, wherein:
transmitting the signal of the first frequency comprises transmitting the signal at a frequency between about 1,000 Hz to about 20,000 Hz; and
transmitting the signal of the second frequency comprises transmitting the signal at a frequency between about 1,000 Hz to about 20,000 Hz, wherein the first frequency is different from the second frequency.

11. The method of claim 1, wherein the beat signal has a frequency within a range of more than 0 Hz to about 5,000 Hz.

12. The method of claim 1, further comprising:
positioning the implantable electrodes to space proximate to nervous tissue of the subject.

13. The method of claim 1, further comprising:
positioning the implantable electrodes to space proximate to vertebral nerves of the subject.

14. The method of claim 1, further comprising:
positioning the implantable electrodes to space proximate to spinal nerves and spinal nerve roots of the subject.

15. The method of claim 1, further comprising:
positioning the implantable electrodes to space proximate to peripheral nerves of the subject.

16. The method of claim 1, further comprising:
positioning the implantable electrodes to space proximate sympathetic and parasympathetic nerves.

17. A method for electrical stimulation of a subject, the method comprising:
transmitting a signal of a first frequency through a first circuit created between a first pair of implantable electrodes positioned in the subject, wherein the first circuit generates a first electrical field;
transmitting a signal of a second frequency through a second circuit created between a second pair of implantable electrodes positioned in the subject, wherein the second circuit generates a second electrical field;
wherein the first pair of implantable electrodes and the second pair of implantable electrodes are positioned in a substantially linear configuration along a same axis such that the first electrical field and the second electrical field are in an axial bias configuration, and
wherein the first electrical field and the second electrical field interfere with each other at an area of overlap to produce a beat signal.

18. The method of claim 17, wherein the first pair of implantable electrodes and the second pair of implantable electrodes are aligned vertically along a longitudinal axis of a spinal cord to form the first circuit and the second circuit and the first circuit is positioned on the same axis as the second circuit.

19. The method of claim 17, wherein the first pair of implantable electrodes and the second pair of implantable electrodes are included on a single lead, wherein the first pair of implantable electrodes and the second pair of implantable electrodes are independently controllable to be arranged as positive and negative electrode pairs for creation of the first circuit and the second circuit.

20. The method of claim 19, wherein the single lead includes a plurality of electrodes arranged in a linear electrode array, and the method further comprises:
altering selection of a first electrode of the first pair of implantable electrodes on the single lead to alter a longitudinal positioning of the beat signal.

21. The method of claim 17, wherein the first pair of implantable electrodes are included on a first lead and the second pair of implantable electrodes are included on a second lead, wherein the first lead and the second lead are positioned in the substantially linear configuration approximately end-to-end, wherein a distance, measured perpendicular to the same axis, between the first lead and the second lead is less than about 2 mm.

22. The method of claim 17, wherein the substantially linear configuration approximately end-to-end comprises at least one electrode on the first lead positioned adjacent to at least one electrode on the second lead.

23. The method of claim 17, wherein:
transmitting the signal of the first frequency comprises transmitting the signal at a frequency between about 1,000 Hz to about 20,000 Hz; and
transmitting the signal of the second frequency comprises transmitting the signal at a frequency between about 1,000 Hz to about 20,000 Hz, wherein the first frequency is different from the second frequency.

24. The method of claim 17, wherein the beat signal has a frequency within a range of more than 0 Hz to about 5,000 Hz.

25. The method of claim 17, further comprising:
positioning the first pair of implantable electrodes to a dura matter in an epidural space proximate to a spinal cord of the subject; and
positioning the second pair of implantable electrodes to the dura matter in the epidural space proximate to the spinal cord of the subject.

26. The method of claim 17, further comprising:
positioning the first pair of implantable electrodes and positioning the second pair of implantable electrodes to space proximate to nervous tissue of the subject.

27. The method of claim 17, further comprising:
positioning the first pair of implantable electrodes and positioning the second pair of implantable electrodes to space proximate to vertebral nerves of the subject.

28. The method of claim 17, further comprising:
positioning the first pair of implantable electrodes and positioning the second pair of implantable electrodes to space proximate to spinal nerves and spinal nerve roots of the subject.

29. The method of claim 17, further comprising:
positioning the first pair of implantable electrodes and positioning the second pair of implantable electrodes to space proximate to Vagus nerves of the subject.

30. An electrical stimulator for electrical stimulation of a subject, comprising:
an interferential current generator which generates an interferential alternating current output comprising first signals and second signals; and
multiple circuits created using implantable electrodes, wherein the implantable electrodes have a first end and a second end, wherein the first ends are coupled to the interferential current generator and the second ends are configured to be positioned in the subject;
wherein the first signals are transmitted through a first circuit of the multiple circuits to generate a first electrical field,
wherein the second signals are transmitted through a second circuit of the multiple circuits to generate a second electrical field,
wherein the implantable electrodes are positioned in a substantially linear configuration along a same axis such that the first electrical field of the first circuit and the second electrical field of the second circuit are in an axial bias configuration, and
wherein the first electrical field and the second electrical field interfere with each other at an area of overlap to produce a beat signal.

31. The electrical stimulator of claim 30, wherein the implantable electrodes include a first pair of implantable electrodes and a second pair of implantable electrodes positioned in the substantially linear configuration along the same axis.

32. The electrical stimulator of claim 30, wherein the multiple circuits include the first circuit between a first implantable electrode and a second implantable electrode, and the second circuit between the first implantable electrode and a third implantable electrode, wherein the first circuit and the second circuit have a common implantable electrode.

33. The electrical stimulator of claim 30, wherein the implantable electrodes are included on a single lead, wherein the implantable electrodes are independently controllable to be arranged as positive and negative electrode pairs for creation of the first circuit and the second circuit.

34. The electrical stimulator of claim 30, wherein the implantable electrodes are included on a first lead and a second lead, wherein the first lead and the second lead are positioned in the substantially linear configuration approximately end-to-end, wherein a distance, measured perpendicular to the same axis, between the first lead and the second lead is less than about 2 mm.
